(19) United States
(12) Patent Application Publication
(10) Pub. No.: US 2017/0209298 A1
(43) Pub. Date: Jul. 27, 2017

(54) PORTABLE URINATION STATION

(71) Applicant: Jason Hastings, Richmond, KY (US)

(72) Inventor: Jason Hastings, Richmond, KY (US)

(21) Appl. No.: 15/006,237

(22) Filed: Jan. 26, 2016

(51) Int. Cl. A61F 5/453; A47K 11/12; A61F 5/455

(52) U.S. Cl. CPC A61F 5/453; A61F 5/455; A47K 11/12

(57) ABSTRACT

A portable urination station including: a receptor; a tube attached to the receptor at a first end; a urine pouch attached to the tube at a second end of the tube; and an inner absorbent material within the urine pouch, where the absorbent material is adapted to receive and hold urine. The receptor may be configured as a male receptor or a female receptor.

### BACKGROUND OF THE INVENTION

Field of Invention

The present invention relates to a device used to provide a portable urination station.

Description of Related Art

Many times travelers may commute long distances in a motor vehicle. In particular, truckers transport and haul goods over long distances on a regular basis. Further individuals may commute significant distances and occasionally may be caught in significant traffic jams. In either situation a driver may be forced or placed in a situation, where they must relief themselves due to time spent within a vehicle. Sometimes it's not practical or possible to quickly leave the highway and find a restroom in order to urinate. As such, in an emergency a driver or in certain instances a passenger will have an urgent need to relief themselves while sitting in traffic or on an extended long distance commute. As a result, it would be advantageous to have a device that could be used within the vehicle so that the driver or passenger is able to release urine into a contained pouch in an emergency situation. Such a portable device could be very convenient and effective for any driver or passenger engaging in an extended commute.

### SUMMARY OF THE INVENTION

The present invention relates to a portable urination station including: a receptor; a tube attached to the receptor at a first end; a urine pouch attached to the tube at a second end of the tube; and an inner absorbent material within the urine pouch, where the absorbent material is adapted to receive and hold urine. The receptor may be configured as a male receptor or a female receptor.

### BRIEF DESCRIPTION OF DRAWINGS

FIG. 1 depicts a urination station in accordance with the present invention.

FIG. 2 depicts an absorbent pad used in conjunction with the urination station according to the present invention.

FIG. 3 depicts a second embodiment of the urination station in accordance with the present invention.

### DETAILED DESCRIPTION

The present invention relates to a portable urination station that provides a portable device for a driver or passenger to urinate therein. The urination station according to the present invention includes a receptor connected to a tube at the first end, where a urine pouch is connected to a second end via tube. The receptor may be configured for a either a female user or male user. Two distinct configurations of the present invention are provided based upon the gender of the user. The pouch according to the present invention is sealed with a zipper and includes an absorbent material within the pouch to absorb urine that travels from the receptor through a tube into the pouch. This absorbent material preferably is a non-woven material similar to use with a diaper. As a result, a user has a convenient device handy that allows the user to release urine in a contained device while traveling in a motor vehicle otherwise.

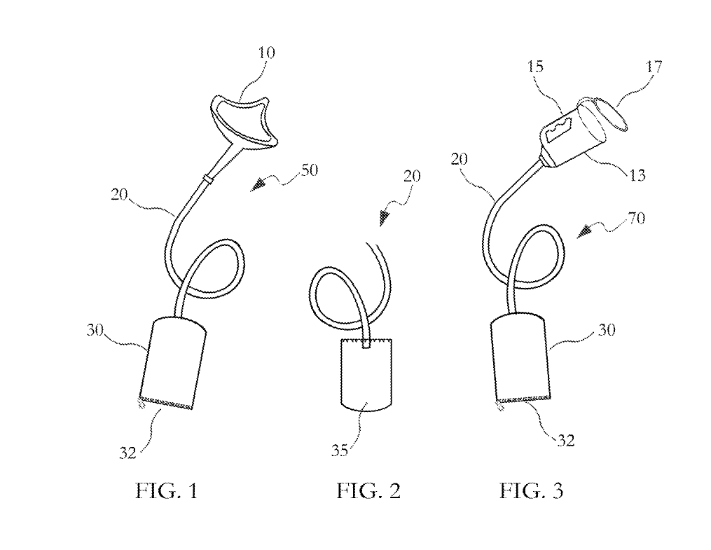

With respect to FIG. 1 a first embodiment of a urination station according to the present invention is depicted. This first embodiment of urination station 50 includes a receptor 10 connected to a tube 20. The receptor 10, as shown in FIG. 1, provides a receptor conformed and configured for use by a female. At the second end of the tube 20 is a pouch 30 that receives urine travelling from the receptor 10 through tube 20 and then into pouch 30. The pouch 30 further includes a zipper 32 that enables a user to remove and change any absorbent material within the pouch 30.

Preferably the pouch 30 includes an absorbent material 35 as shown in FIG. 2. This absorbent material 35 absorbs urine traveling through tube 20 and therefore contains the urine within the pouch 30 until a user deposes of the absorbent material 35 and/or pouch 30 as needed.

FIG. 3 depicts an alternative embodiment suitable for use by a male user. A male receptor 15 shown in FIG. 3 includes a main portion 13 with a lid 17. The male receptor 15 has an opening revealed upon lifting lid 17 and is somewhat symmetrical in shape. The female receptor 10 is conformed so that a female user may place the receptor against the skin surrounding the genitals of the female user. Therefore either receptor is suitable and adapted for use each gender.

The urination station according to the present invention therefore provides a suitable means for collecting urine to limit any spilling or leakage while travelling. Further it also helps individuals such as truckers to avoid using messy or non-hygienic alternatives to relieving themselves such as large container or bottle. The instant invention has been shown and described in what it considers to be the most practical and preferred embodiments. It is recognized, however, that departures may be made there from within the scope of the invention and that obvious modifications will occur to a person skilled in the art.

1. A portable urination station comprising:
a. a receptor;
b. a tube attached to the receptor at a first end;
c. a urine pouch attached to the tube at a second end of the tube, wherein the urine pouch is sealed with a zipper; and
d. an inner absorbent non-woven material within the urine pouch, where the absorbent non-woven material is adapted to receive and hold urine and wherein the inner absorbent non-woven material is disposable.

2. The portable urination station according to claim 1, where the receptor is a male receptor.

3. The portable urination station according to claim 1, where the receptor is a female receptor.
